(19) United States
(12) Patent Application Publication
(10) Pub. No.: US 2023/0306303 A1
(43) Pub. Date: Sep. 28, 2023

(54) Systems and Methods for Crowdsourced Machine Learning

(71) Applicant: The Board of Trustees of the Leland Stanford Junior University, Stanford, CA (US)

(72) Inventors: Dennis Paul Wall, Palo Alto, CA (US); Peter Yigitcan Washington, Stanford, CA (US)

(73) Assignee: The Board of Trustees of the Leland Stanford Junior University, Stanford, CA (US)

(21) Appl. No.: 18/020,907

(22) PCT Filed: Aug. 11, 2021
(86) PCT No.: PCT/US2021/045613

§ 371 Date: Feb. 10, 2023

Related U.S. Application Data
(60) Provisional application No. 63/230,005, filed on Aug. 5, 2021.
(60) Provisional application No. 63/064,380, filed on Aug. 11, 2020.

(51) Int. Cl. G06N 20/00; G16H 50/20

(52) U.S. Cl. CPC G06N 20/00; G16H 50/20

(57) ABSTRACT

Systems and methods for crowdsourced machine learning in accordance with embodiments of the invention are illustrated. In many embodiments, particular crowdworkers from a plurality of crowdworkers who are able to perform with high accuracy and reliability (referred to herein as “super recognizers”) are identified and used to generate training data for machine learning models. In various embodiments, super recognizers are identified by providing a request to answer questions regarding a particular type of input to the plurality of crowdworkers and providing received answers to a machine learning model trained using expert-annotated inputs similar to the inputs provided to the crowdworkers.

### CROSS-REFERENCE TO RELATED APPLICATIONS

The current application is a U.S. National Stage Patent Application which claims priority to PCT Patent Application No. PCT/US2021/045613 titled “Systems and Methods for Crowdsourced Machine Learning”, filed Aug. 11, 2021, which claims the benefit of and priority under 35 U.S.C. § 119(e) to U.S. Provisional Patent Application No. 63/064,380 titled “Precision Telemedicine through Crowdsourced Machine Learning: Testing Variability of Crowd Workers for Video-Based Autism Feature Recognition”, filed Aug. 11, 2020, and U.S. Provisional Patent Application No. 63/230,005 titled “Systems and Methods for Crowdsourced Machine Learning” filed Aug. 5, 2021. The disclosure of PCT Patent Application No. PCT/US2021/045613 and U.S. Provisional Patent Application Nos. 63/064,380 and 63/230,005 are hereby incorporated by reference in its entirety.

### STATEMENT OF FEDERALLY SPONSORED RESEARCH

This invention was made with Government support under contracts EB025025 and HD091500 awarded by the National Institutes of Health. The Government has certain rights in the invention.

### FIELD OF THE INVENTION

The present invention generally relates to systems and methods for crowdsourcing machine learning, namely the automated identification of quality training data annotators for use in training machine learning models.

### BACKGROUND

Machine learning is a field of computer science concerned with models that improve automatically through experience. There are many different types of machine learning models. Supervised learning is the machine learning task of learning a function that maps an input to an output based on example input-output pairs. These input-output pairs are often referred to as “training data.” The accuracy of a supervised learning model is often determined at least in part by the quality of the training data. That is, the more accurate the outputs are to their respective inputs, the more accurate the machine learning model trained using those pairs.

Crowdsourcing is the practice of obtaining goods or services by enlisting the services of a large number of people. Crowdsourcing platforms such as Amazon Mechanical Turk by Amazon.com, Inc., provide the ability to post tasks that individuals can perform remotely for a predetermined fee.

### SUMMARY OF THE INVENTION

Systems and methods for crowdsourced machine learning in accordance with embodiments of the invention are illustrated. One embodiment includes a method for crowdsourced machine learning, including obtaining an evaluation data set comprising a plurality of inputs and a plurality of outputs, where each output in the plurality of outputs uniquely corresponds to an input in the plurality of inputs, and where each output is assumed to accurately label its corresponding input, providing the plurality of inputs to a plurality of crowdworkers, receiving a plurality of annotations from each crowdworker in the plurality of crowdworkers, where each plurality of annotations includes an annotation for at least one input in the plurality of inputs, calculating at least one confidence metric for each crowdworker based on the plurality of annotations received from each crowdworker, identifying a plurality of super recognizers from the plurality of crowdworkers based on the at least one confidence metric associated with the at least one crowdworker, obtaining an unlabeled data set, providing the unlabeled data set to each crowdworker in the plurality of super recognizers, receiving a second plurality of annotations from each crowdworker in the plurality of super recognizers, aggregating the second plurality of annotations, generating a training data set by merging the aggregated second plurality of annotations and the unlabeled data set, and training a machine learning model using the generated training data set.

In another embodiment, the at least one confidence metric is the probability of correct classification (PCC) of a pretrained machine learning model.

In a further embodiment, the PCC is calculated for a given crowdworker in the plurality of crowdworkers by providing a recognizer machine learning model with a given plurality of annotations for the plurality of inputs generated by the given crowdworker.

In still another embodiment, the recognizer machine learning model is a binary logisitic regression classifier.

In a still further embodiment, the at least one confidence metric is selected from the group consisting of: a test-retest metric, a reliability metric, a penalized time metric, and a time spent metric.

In yet another embodiment, the plurality of inputs are provided to a plurality of crowdworkers in response to crowdworkers in the plurality of crowdworkers responding to a request on a crowdsourcing platform.

In a yet further embodiment, the plurality of crowdworkers includes crowdworkers who have completed one or more requests.

In another additional embodiment, the plurality of inputs and the unlabeled data set both includes anonymized videos of children with Autism Spectrum Disorder (ASD).

In a further additional embodiment, the plurality of outputs, the plurality of annotations, and the second plurality of annotations includes responses to a questionnaire.

In another embodiment again, the machine learning model is trained to identify ASD in videos of children.

In a further embodiment again, the plurality of inputs and the unlabeled dataset is modified to protect privacy.

In still yet another embodiment, a crowdsourced machine learning device, includes a processor; and a memory, the memory containing a crowdsourced machine learning application capable of direction the processor to, obtain an evaluation data set comprising a plurality of inputs and a plurality of outputs, where each output in the plurality of outputs uniquely corresponds to an input in the plurality of inputs, and where each output is assumed to accurately label its corresponding input, providing the plurality of inputs to a plurality of crowdworkers via a crowdsourcing platform, receive a plurality of annotations from each crowdworker in the plurality of crowdworkers via the crowdsourcing platform, where each plurality of annotations includes an annotation for at least one input in the plurality of inputs, calculate at least one confidence metric for each crowdworker based on the plurality of annotations received from each crowdworker, identify a plurality of super recognizers from the plurality of crowdworkers based on the at least one confidence metric associated with the at least one crowdworker, obtain an unlabeled data set, provide the unlabeled data set to each crowdworker in the plurality of super recognizers via the crowdsourcing platform, receive a second plurality of annotations from each crowdworker in the plurality of super recognizers via the crowdsourcing platform, aggregate the second plurality of annotations, generate a training data set by merging the aggregated second plurality of annotations and the unlabeled data set, and train a machine learning model using the generated training data set.

In a still yet further embodiment, the at least one confidence metric is the probability of correct classification (PCC) of a pretrained machine learning model.

In still another additional embodiment, the PCC is calculated for a given crowdworker in the plurality of crowdworkers by providing a recognizer machine learning model with a given plurality of annotations for the plurality of inputs generated by the given crowdworker.

In a still further additional embodiment, the recognizer machine learning model is a binary logisitic regression classifier.

In still another embodiment again, the at least one confidence metric is selected from the group consisting of: a test-retest metric, a reliability metric, a penalized time metric, and a time spent metric.

In a still further embodiment again, the plurality of inputs are provided to a plurality of crowdworkers in response to crowdworkers in the plurality of crowdworkers responding to a request on a crowdsourcing platform.

In yet another additional embodiment, the plurality of crowdworkers includes crowdworkers who have completed one or more requests.

In a yet further additional embodiment, the plurality of inputs and the unlabeled data set both includes anonymized videos of children with Autism Spectrum Disorder (ASD).

In yet another embodiment again, the plurality of outputs, the plurality of annotations, and the second plurality of annotations includes responses to a questionnaire.

In a yet further embodiment again, the machine learning model is trained to identify ASD in videos of children.

In another additional embodiment again, the plurality of inputs and the unlabeled dataset is modified to protect privacy.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 is a system diagram for a crowdsourced machine learning system in accordance with an embodiment of the invention.

FIG. 2 is a block diagram of a crowdsourced machine learning device in accordance with an embodiment of the invention.

FIG. 3 is a flow chart illustrating a process for crowdsourced machine learning in accordance with an embodiment of the invention.

FIG. 4 is a flow chart illustrating another process for crowdsourced machine learning in accordance with an embodiment of the invention.

FIG. 5 is a graphical illustration of yet another process for crowdsourced machine learning in accordance with an embodiment of the invention.

### DETAILED DESCRIPTION

Turning now to the drawings, systems and methods for crowdsourced machine learning are described. A considerable problem for the field of machine learning is the acquisition of high-quality training data. In order to be accurate across different inputs, many machine learning models are reliant on large volumes of accurate training data. However, it is often difficult and/or expensive to acquire training data in sufficient volume and/or accuracy to train a model to sufficient accuracy. In many circumstances, small training data sets are synthetically enhanced by slightly modifying the available original training data set to produce more instances. In some circumstances, for example in health-related fields, natural language processing is used to try and merge different data sets into a single training data set. However, both of these conventional methodologies are flawed in that they rely on unreliable computational assumptions, i.e. in the first case there is rarely proof the synthetic data is reliable or useful, and in the second case there is often no way but extensive manual revision to ensure that the merge was completed reliably.

Systems and methods described herein resolve this issue by generating natural, high volume, high accuracy training data sets through crowdsourcing. That said, crowdsourcing traditionally relies on sending tasks to essentially random persons with little ability to verify their ability to perform reliably. As such, systems and methods described herein enable the identification of “super recognizers” in the crowdworker population who are specific crowdworkers that produce reliably accurate results. Super recognizers can in turn be used to generate high volumes of training data which can be used to train machine learning models. An advantage of using crowdsourcing is that the job can be performed by anyone in any part of the world with access to the Internet, and therefore a much larger talent pool can be relied upon, often at lower cost. Crowdsourced machine learning systems are described first below.

### Crowdsourced Machine Learning Systems

Crowdsourced machine learning systems are capable of identifying super recognizers in a pool of crowdworkers and engaging their expertise to generate training data. The training data can then be used to train machine learning models as appropriate to the requirements of specific applications of embodiments of the invention. In some embodiments, the training data includes as inputs videos of children, and outputs as classifications as to whether or not the individual presents with a specific set of behavioral features, or visually or audially recognizable features, or conditions that can be recognized through the appearance (audio and or visually recognized) of sets of features of Autism Spectrum Disorder (ASD, or Autism). However, any number of different training data can be generated, depending on the model to be trained. In various embodiments, the crowdsourced machine learning system uses a small amount of training data to identify super recognizers, which in turn are used to generate more training data.

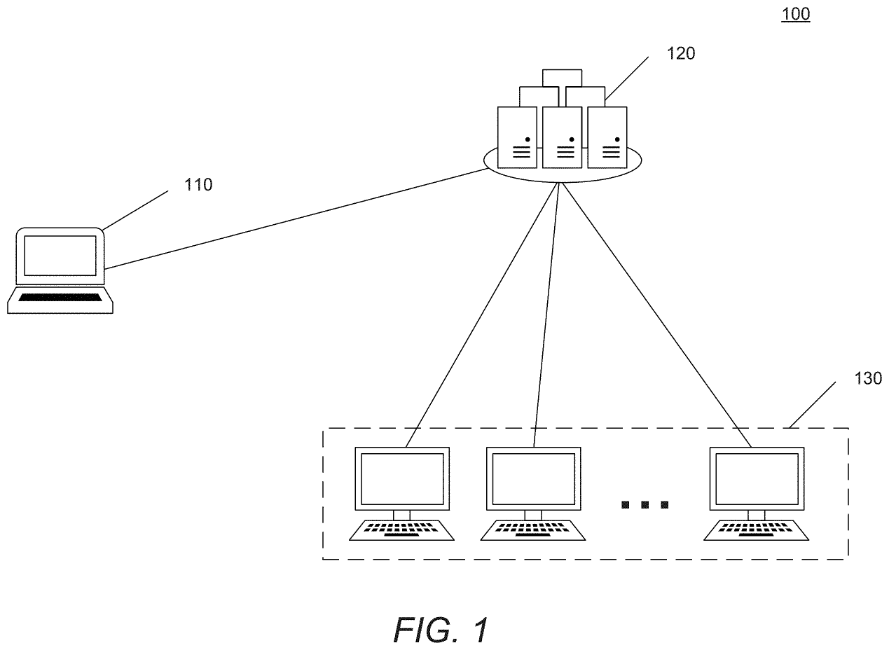

Turning now to FIG. 1, a crowdsourced machine learning system in accordance with an embodiment of the invention is illustrated. System 100 includes a crowdsourced machine learning device 110. In many embodiments the crowdsourced machine learning device is a personal computer, however any number of different types of computing device such as (but not limited to) servers, smart phones, cloud computing clusters, tablet computers, and/or any type of computing device can be used as appropriate to the requirements of specific applications of embodiments of the invention. Crowdsourced machine learning devices can be used to acquire a base set of training data for use in identifying super recognizers, and can subsequently identify those super recognizers. In numerous embodiments, crowdsourced machine learning devices can train machine learning models using training data obtained from super recognizers.

Crowdsourced machine learning device 100 is communicatively linked to a crowdsourcing platform 120. Crowdsourcing platforms are capable of receiving requests and providing an interface by which crowdworkers can fulfill the request, often in exchange for compensation. In numerous embodiments, the crowdsourcing platform can be (but is not limited to) Amazon Mechanical Turk by Amazon.com, Inc. In numerous embodiments, the communicative link is established via the Internet, however any number of different networks, both wired and wireless, and/or alone or in combination with other networks can be used to establish the communicative link. In various embodiments, the crowdsourced machine learning device can transmit request to the crowdsourcing platform that can include data necessary to complete the request. In many embodiments, the crowdsourcing platform is implemented using one or more servers. However, they can be implemented using smaller scale computing architectures.

The crowdsourcing platform 120 can distribute the request to any number of crowdworker devices 130. Crowdworker devices are any computational device that can receive requests from a crowdsourcing platform and enable the crowdworker to complete the request. In many embodiments, crowdworker devices can include (but are not limited to) personal computers, smart phones, tablet computers, smart TVs, smart watches, and/or any other computing device as appropriate to the requirements of specific applications of embodiments of the invention. In numerous embodiments, the crowdsourcing platform communicates with crowdworker devices via the Internet, although as above, any network configuration can be used. Crowdworker devices can be identified with a specific crowdworker. While a specific system architecture is illustrated with respect to FIG. 1, any number of different architectures (e.g. those that use multiple crowdsourced machine learning devices, and/or different communicative links) can be used as appropriate to the requirements of specific applications of embodiments of the invention.

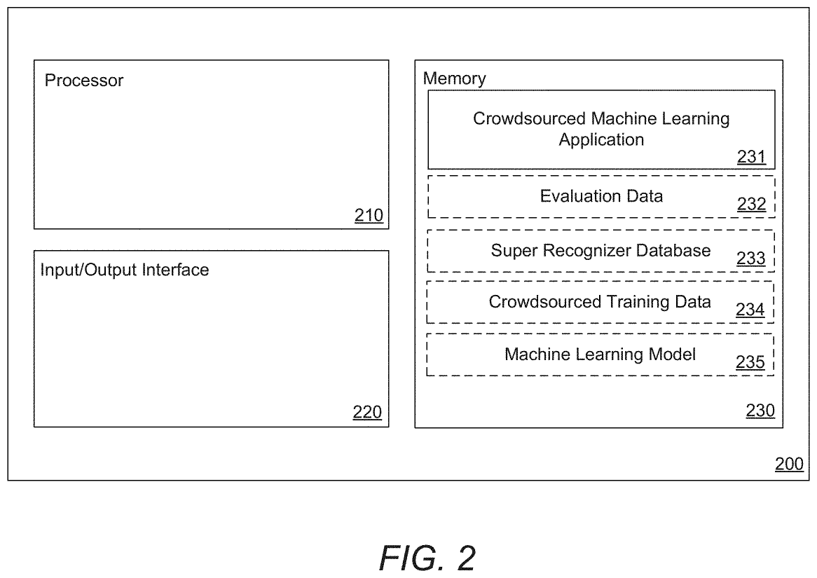

Turning now to FIG. 2, a block diagram for a crowdsourced machine learning device in accordance with an embodiment of the invention is illustrated. Crowdsourced machine learning device 200 includes a processor 210. Processor 210 can be any logic processing circuitry capable of performing crowdsourced machine learning processes. In many embodiments, processors are central processing units (CPUs), graphics processing units (GPUs), field-programmable gate arrays (FPGAs), application-specific integrated circuits (ASICs), and/or any other logic circuit and/or combination thereof as appropriate to the requirements of specific applications of embodiments of the invention.

Device 200 further includes an input/output (I/O) interface 220. I/O interfaces are capable of transmitting data from crowdsourced machine learning devices to crowdsourcing platforms and/or any other computing device as appropriate to the requirements of specific applications of embodiments of the invention. Device 200 further includes a memory 230 which contains a crowdsourced machine learning application 231. Crowdsourced machine learning applications are capable of directing the processor to carry out various crowdsourced machine learning processes which are discussed in further detail below.

In many embodiments, the memory 200 may variously contain: evaluation data 232, super recognizer database 233, crowdsourced training data 234, and machine learning model 235. Evaluation data is data that has been confirmed as an accurate (“gold standard”). In many embodiments, the evaluation data is validated by experts and can be assumed to be an accurate set of classifications. Evaluation data can include a number of input-output pairs (also referred to as “records”), where the input is what the machine learning model to be trained takes as an input, and the output is what the machine learning model should output based on the associated input. For example, if the input is a video of a child, the output may be a metric indicating whether or not the child presents with ASD. However as can be readily appreciated, the inputs and outputs can be any pair of data and classification as appropriate to the requirements of specific applications of embodiments of the invention. In many embodiments, evaluation data and/or any data that is transmitted to crowdworkers is anonymized to protect privacy. Super recognizer databases are databases of crowdworkers that have been identified as super recognizers. Super recognizer databases can be constructed to store one or more unique identifiers for each super recognizer, such as (but not limited to) account identifiers for accounts belonging to super recognizers on a crowdsourcing platform. Crowdsourced training data is training data that has been obtained from super recognizers. The machine learning model can be any machine learning model to be trained using the crowdsourced training data.

While a specific architecture for a crowdsourced machine learning device is illustrated in FIG. 2, any number of different computational architectures can be used as appropriate to the requirements of specific applications of embodiments of the invention. Further, as can be readily appreciated, evaluation data 232, super recognizer database 233, crowdsourced training data 234, and machine learning model 235 need not be present at the same time, or at all as appropriate to the requirements of specific applications of embodiments of the invention. Crowdsourced machine learning processes are discussed in further detail below.

### Crowdsourced Machine Learning Processes

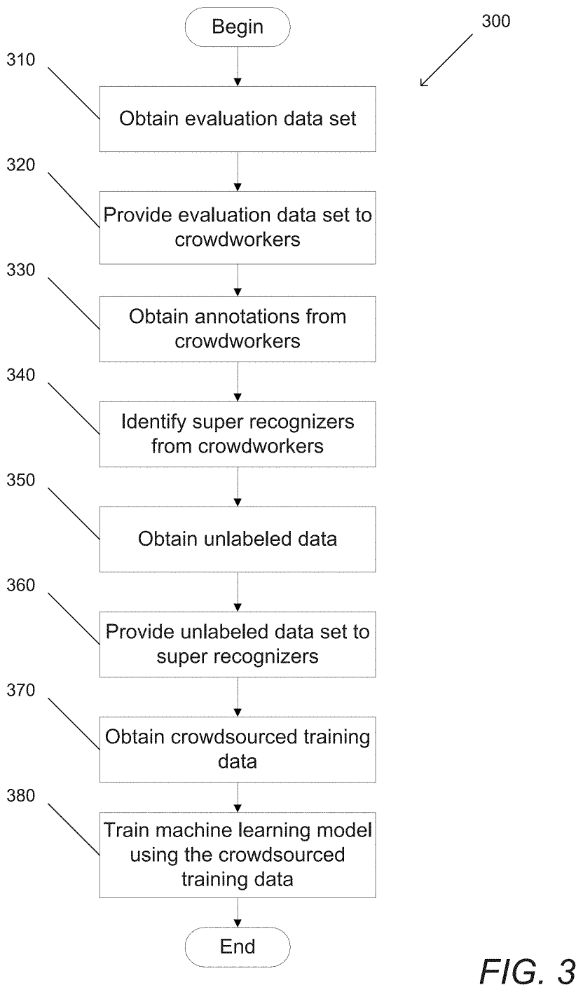

Crowdsourced machine learning processes involve identifying super recognizers and utilizing them to quickly and relatively inexpensively generate a large volume of accurate crowdsourced training data. The crowdsourced training data can then be used to train machine learning models. In many embodiments, super recognizers can be identified through providing them requests to classify data and evaluating their performance. In various embodiments, evaluation of super recognizer performance is achieved by comparing their performance to one or more confidence metrics. Turning now to FIG. 3, a flow chart for a crowdsourced machine learning process in accordance with an embodiment of the invention is illustrated.

Process 300 includes obtaining (310) an evaluation data set. The inputs of the evaluation data set (i.e. an unlabeled version of the evaluation data set) are provided (320) to crowdworkers via a broad request. In many embodiments, while the request is broad, there may restrictions on which crowdworkers can accept the request in order to recruit trustworthy and capable workers. For example, in many embodiments, only workers who have performed a minimum number of requests can accept the job. Annotations of the unlabeled version of the evaluation data set generated by crowdworkers are obtained (330). In many embodiments, instead of annotations that include classifications which would be representative of the actual output component of the evaluation data, a set of answers to questions about the inputs which can be processed to produce a classification are obtained (330). In continuation of the previous example, a clinically relevant questionnaire about the behavior of a child in a video can be obtained, the answers of which may reflect a diagnosis of ASD.

Of the crowdworkers that responded, a subset of the crowdworkers are identified (340) as super recognizers. In many embodiments, crowdworkers that produced identical labels to those found in the evaluation data set are identified as super recognizers. In many embodiments, crowdworkers are evaluated using a machine learning model. For example, in various embodiments, a binary logistic regression classifier can be trained using the evaluation data and/or a separate corpus of “gold standard” training data. In numerous embodiments, the output of the binary logistic regression classifier is a probability for a binary outcome, which can be treated as a confidence score of a crowdworker's response when the classifier is provided with the crowdworker's response. A crowdworker's mean probability of a correct classification (PCC) across a number of different input-output pairs can be computed, and those with mean PCC's greater than a threshold value can be identified as super recognizers. In many embodiments, a mean PCC greater than 75% is sufficient to be identified as a super recognizer. However, as can be readily appreciated, depending on the level of accuracy needed for the crowdsourced training data, this value can be modified upwards (for a higher probability of accuracy over time, or conversely lower). In many embodiments, a record of each super recognizer can be stored in a database. However, any number of different machine learning models can be used with similar effect to evaluate crowdworkers.

Further, other metrics can be considered when identifying super recognizers. In many embodiments, crowdworkers can be provided the same task (i.e. using the same exact data) at least one month apart, without being provided their previously submitted annotations or flagging to them that it is a repeat test. The mean deviation from the previous answers can be recorded as a metric of a given crowdworker's test-retest reliability. Higher values for this metric correspond to greater variation in responses when re-testing on the same data.

Reliability of an individual crowdworker's answers across different records in a data set can be calculated as the mean L1 distance between a given annotation to a single record compared to all of the records they annotated. This mean pairwise internal L1 distance is high when workers provide a wide variety of answer patterns across records. If the answer was the same for each record, the value would be 0.

In many embodiments, metrics describing more efficient crowdworkers can be calculated in order to identify super recognizers. Indeed, if it takes a long time for an individual crowdworker to achieve satisfactory results, they may not be worth engaging in the future. Conversely, if a worker is spending to little time, it may indicate that they are not paying attention and merely trying to rush through the task in order to get compensated. A penalized time (PT) metric can be calculated that rewards workers who spend sufficient time annotating the first time while rewarding (often to a lesser extent) workers who spend sufficient time rating after receiving a timeliness warning. Workers who either do not spend more time rating after receiving a warning or who do not sufficiently update their answers can also be penalized. The PT metric then functions as a measure of trustworthiness. In many embodiments, if a crowdworker spends longer than a time threshold T rating, they are not asked to revise their answers and receive a baseline score M. If they do not spend a sufficient time (T) annotating, then they are asked to spend more time and to revise their answers. In this case, the PT metric consistes of two terms balanced by a weighting constant, c. The first term is the revision test-retest reliability (RMSCL1) between initial and revised answers only for the record that the crowdworker was asked to revise. The second term is the mean of total time spent annotating, which is the time spent initially (t1) and the time spent revising (t2). This is formalized as:

$$PT = \{ \begin{matrix}M , & t 1 \geq T \\ \frac{t 1 + t 2}{N} cRMSCL 1 , & t 1 < T\end{matrix}$$

In many embodiments, the time spent annotating per record is recorded. These metrics of a crowdworker's performance can be used to further filter which crowdworkers are identified as super recognizers as appropriate to the requirements of specific applications of embodiments of the invention.

Process 300 further includes obtaining (350) unlabeled data. In many embodiments, the unlabeled data is of the same (or approximately the same) type as the inputs of the evaluation data set. The unlabeled data is provided (360) to identified super recognizers as part of a request which can be accepted by said super recognizers. Crowdsourced training data is obtained (370) by receiving annotations from super recognizers. In many embodiments, the annotations are aggregated and merged with the unlabeled to form input-output pairs. In many embodiments, when the same records are provided to multiple crowdworkers, a consensus as to which annotations will be accepted is achieved. In numerous embodiments, the consensus is achieved by selecting the majority label in the set of received annotations for a particular record. In various embodiments, a soft-target probability distribution label is calculated base on the received annotations for the particular record. The crowdsourced training data can then be used to train (380) a machine learning model.

As can be readily appreciated, FIG. 3 illustrates a general process for crowdsourced machine learning, and the process can be modified as appropriate to the requirements of specific applications of embodiments of the invention. For example, multiple different rounds of identification may occur on the same crowdworkers with multiple sets of evaluation data (or subsets of an evaluation data set). By way of further example, in many embodiments, steps are performed asynchronously and/or repetitively. That is, additional super recognizers may continue to be identified over time in response to the evaluation data set request. Further, the process may be modified depending on the specifics of the training data to be produced.

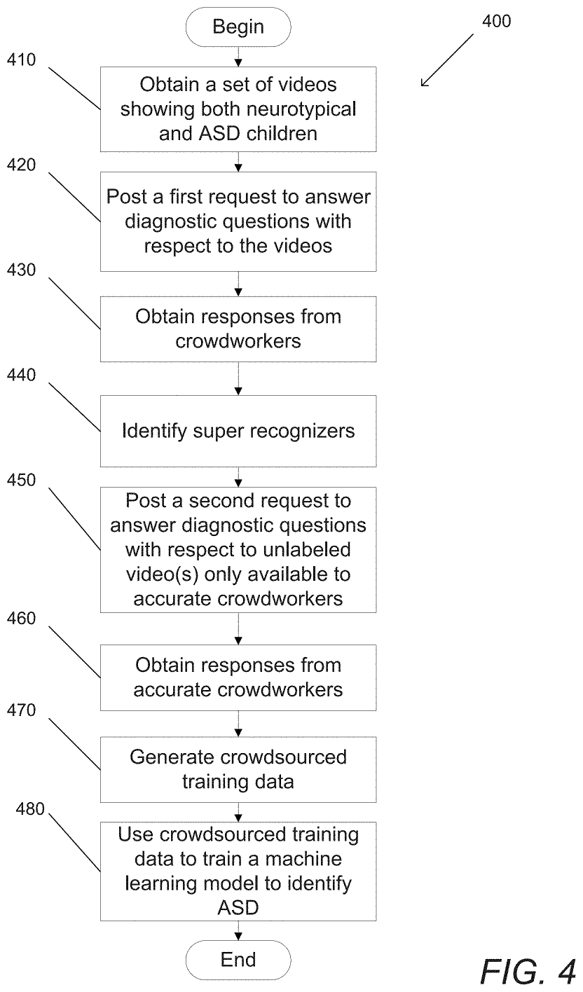

To illustrate this, turning now to FIG. 4, a flow chart for a crowdsourced machine learning process for training a machine learning model to identify ASD in children in accordance with an embodiment of the invention is illustrated. Process 400 includes obtaining (410) a set of videos showing both neurotypical children and children presenting with ASD. In many embodiments, the videos represent the input, and the classification of neurotypical or ASD represents the output of a training data set. In numerous embodiments, the videos represent the input, while the output is a set of answers to questions in a questionnaire about the videos relevant to relevant to diagnosing ADS. A first request to fill out the questionnaire for a minimum number of different videos is posted (420) to a crowdsource platform. In many embodiments, a crowdworker is instructed to complete the questionnaire for a minimum number of videos as a precaution against luck. In numerous embodiments, the minimum number is 10 or more, but this threshold may change depending on the complexity of the task.

Responses are obtained (430) from the crowdworkers and super recognizers are identified (440) from the responding crowdworkers. In many embodiments, a binary logistic regression classifier is trained clinically validated answers to the questionnaire and/or clinically validated answers to a similar version of the questionnaire where questions in the first and seconded questionnaires are mapped to each other, and run on the responses from the crowdworkers. Those with sufficiently high mean PCC values (e.g. >75%, >75%+1 standard deviation, etc.) can be selected as super recognizers.

A second request is posted (450) to the crowdsourcing platform which requests that ASOD schedules be completed for a number of different unlabeled videos. In many embodiments, the second request can only be accepted by crowdworkers who are identified as being super recognizers. Their accurate responses are obtained (460) and used to generate crowdsourced training data (470). In many embodiments, crowdsourced training data is generated by merging received output responses with the respective provided video input, and all input-output pairs are again merged into a singular crowdsourced training data set. The crowdsourced training data set is used to train (480) a machine learning model to produce ASOD reports for any given input video of a child.

As can be readily appreciated by one of ordinary skill in the art, FIG. 4 represents a specific embodiment, and many different modifications are possible. For example, different methods of classifying ASD and/or identifying super recognizers can be used depending on the type of data to be classified as appropriate to the requirements of specific applications of embodiments of the invention. Further, systems and methods described herein are not limited to recognizing ASD, and indeed can be used for any number of classification tasks such as (but not limited to) identification of any number of neurological conditions, for example attention definicit hyperactive disorder, speech development delays, global developmental disorders, childhood schizophrenia, childhood disintegrative disorder, bipolar disorder, and schizophrenia. As can be readily appreciated, any classification task (not limited to neurological conditions) can be performed by systems and methods described herein.

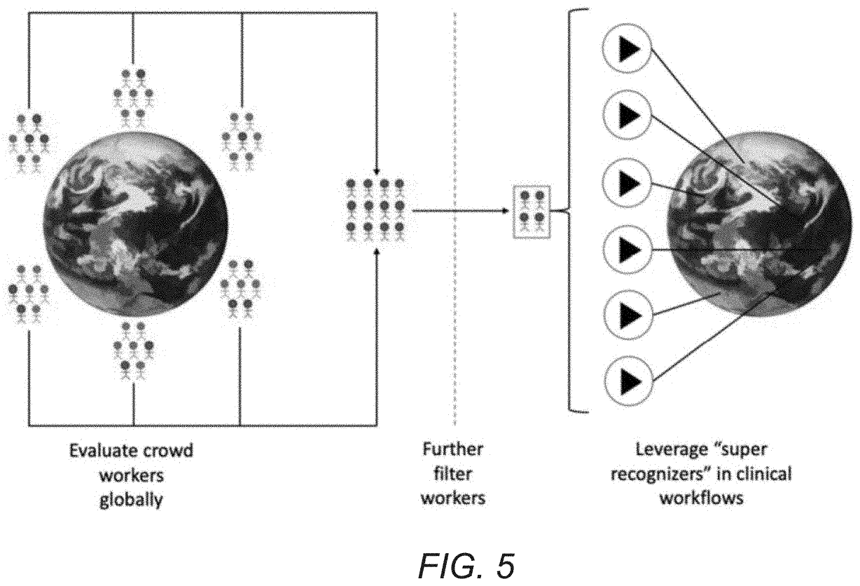

Turning now to FIG. 5, a high-level pictorial representation of a crowdsourced machine learning process in accordance with an embodiment of the invention is illustrated. As can be seen, crowdworkers all over the world can be evaluated until a set of super recognizers are identified which in turn can be leveraged in clinical workflows.

Although specific systems and methods for crowdsourced machine learning are discussed above, many different systems and methods can be implemented in accordance with many different embodiments of the invention. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A method for crowdsourced machine learning, comprising:
obtaining an evaluation data set comprising a plurality of inputs and a plurality of outputs:
where each output in the plurality of outputs uniquely corresponds to an input in the plurality of inputs; and
where each output is assumed to accurately label its corresponding input;
providing the plurality of inputs to a plurality of crowdworkers;
receiving a plurality of annotations from each crowdworker in the plurality of crowdworkers, where each plurality of annotations comprises an annotation for at least one input in the plurality of inputs;
calculating at least one confidence metric for each crowdworker based on the plurality of annotations received from each crowdworker;
identifying a plurality of super recognizers from the plurality of crowdworkers based on the at least one confidence metric associated with the at least one crowdworker;
obtaining an unlabeled data set;
providing the unlabeled data set to each crowdworker in the plurality of super recognizers;
receiving a second plurality of annotations from each crowdworker in the plurality of super recognizers;
aggregating the second plurality of annotations;
generating a training data set by merging the aggregated second plurality of annotations and the unlabeled data set; and
training a machine learning model using the generated training data set.

2. The method of crowdsourced machine learning of claim 1, wherein the at least one confidence metric is the probability of correct classification (PCC) of a pretrained machine learning model.

3. The method of crowdsourced machine learning of claim 2, wherein the PCC is calculated for a given crowdworker in the plurality of crowdworkers by providing a recognizer machine learning model with a given plurality of annotations for the plurality of inputs generated by the given crowdworker.

4. The method of crowdsourced machine learning of claim 3, wherein the recognizer machine learning model is a binary logisitic regression classifier.

5. The method of crowdsourced machine learning of claim 1, wherein the at least one confidence metric is selected from the group consisting of: a test-retest metric, a reliability metric, a penalized time metric, and a time spent metric.

6. The method of crowdsourded machine learning of claim 1, wherein the plurality of inputs are provided to a plurality of crowdworkers in response to crowdworkers in the plurality of crowdworkers responding to a request on a crowdsourcing platform.

7. The method of crowdsourced machine learning of claim 1, wherein the plurality of crowdworkers comprise crowdworkers who have completed one or more requests.

8. The method of crowdsourced machine learning of claim 1, wherein the plurality of inputs and the unlabeled data set both comprise anonymized videos of children with Autism Spectrum Disorder (ASD).

9. The method of crowdsourced machine learning of claim 8, wherein the plurality of outputs, the plurality of annotations, and the second plurality of annotations comprise responses to a questionnaire.

10. The method of crowdsourced machine learning of claim 8, wherein the machine learning model is trained to identify ASD in videos of children.

11. A crowdsourced machine learning device, comprising:
a processor; and
a memory, the memory containing a crowdsourced machine learning application capable of direction the processor to:
obtain an evaluation data set comprising a plurality of inputs and a plurality of outputs:
where each output in the plurality of outputs uniquely corresponds to an input in the plurality of inputs; and
where each output is assumed to accurately label its corresponding input;
providing the plurality of inputs to a plurality of crowdworkers via a crowdsourcing platform;
receive a plurality of annotations from each crowdworker in the plurality of crowdworkers via the crowdsourcing platform, where each plurality of annotations comprises an annotation for at least one input in the plurality of inputs;
calculate at least one confidence metric for each crowdworker based on the plurality of annotations received from each crowdworker;
identify a plurality of super recognizers from the plurality of crowdworkers based on the at least one confidence metric associated with the at least one crowdworker;
obtain an unlabeled data set;
provide the unlabeled data set to each crowdworker in the plurality of super recognizers via the crowdsourcing platform;
receive a second plurality of annotations from each crowdworker in the plurality of super recognizers via the crowdsourcing platform;
aggregate the second plurality of annotations;
generate a training data set by merging the aggregated second plurality of annotations and the unlabeled data set; and
train a machine learning model using the generated training data set.

12. The crowdsourced machine learning device of claim 11, wherein the at least one confidence metric is the probability of correct classification (PCC) of a pretrained machine learning model.

13. The crowdsourced machine learning device of claim 12, wherein the PCC is calculated for a given crowdworker in the plurality of crowdworkers by providing a recognizer machine learning model with a given plurality of annotations for the plurality of inputs generated by the given crowdworker.

14. The crowdsourced machine learning device of claim 13, wherein the recognizer machine learning model is a binary logisitic regression classifier.

15. The crowdsourced machine learning device of claim 11, wherein the at least one confidence metric is selected from the group consisting of: a test-retest metric, a reliability metric, a penalized time metric, and a time spent metric.

16. The crowdsourced machine learning device of claim 11, wherein the plurality of inputs are provided to a plurality of crowdworkers in response to crowdworkers in the plurality of crowdworkers responding to a request on a crowdsourcing platform.

17. The crowdsourced machine learning device of claim 11, wherein the plurality of crowdworkers comprise crowdworkers who have completed one or more requests.

18. The crowdsourced machine learning device of claim 11, wherein the plurality of inputs and the unlabeled data set both comprise anonymized videos of children with Autism Spectrum Disorder (ASD).

19. The crowdsourced machine learning device of claim 18, wherein the plurality of outputs, the plurality of annotations, and the second plurality of annotations comprise responses to a questionnaire.

20. The crowdsourced machine learning device of claim 18, wherein the machine learning model is trained to identify ASD in videos of children.
